(19) United States
(12) Patent Application Publication
(10) Pub. No.: US 2006/0183781 A1
(43) Pub. Date: Aug. 17, 2006

(54) Novel thiazolylmethyl pyrazoles, method for the production thereof, and use thereof in dyes for keratin fibers

(76) Inventors: Otto Goettel, Marly (CH); Hans-Juergen Braun, Ueberstorf (CH)

Correspondence Address: MICHAEL J. STRIKER, 103 EAST NECK ROAD, HUNTINGTON, NY, 11743, US

(21) Appl. No.: 10/542,825

(22) PCT Filed: Feb. 3, 2004
(86) PCT No.: PCT/EP04/00943

§ 371 Date: Dec. 22, 2005

(30) Foreign Application Priority Data
May 27, 2003 (DE) 103-23-970.7

(51) Int. Cl. C07D 277/28; C07D 417/02; A61K 31/427

(52) U.S. Cl. 514/365; 548/205; 548/204

(57) ABSTRACT

4,5-Diaminopyrazoles of general formula (I) 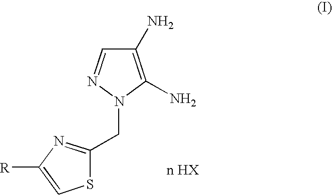
wherein R denotes a straight-chain or branched C₁-C₆-alkyl group, an unsubstituted phenyl group or a singly or multiply substituted phenyl group wherein the substituents are selected independently of each other from among a halogen atom, a C₁-C₆ carboxylate ester group, a straight-chain or branched C₁-C₆-alkoxy group, a straight-chain or branched C₁-C₆-alkoxy group interrupted by one or two hetero atoms, a hydroxyethoxy group, a dihydroxypropoxy group or a nitrile group; furthermore oxidation colorants for keratin fibers containing these compounds and a method for producing said compounds.

The present invention has for an object novel 4,5-diaminopyrazoles and their physiologically compatible salts of organic or inorganic acids. Another object is the preparation of said compounds and the use thereof as dye precursors in oxidative colorants for keratin fibers.

In the field of conventional hair dyeing, oxidation dyes have attained substantial cosmetic significance. In such dyeing, the color is produced by reaction of certain developers with certain couplers in the presence of an oxidant. As in the past, colorants for dyeing hair to natural shades are particularly important. In addition, currently stylish color shades can be produced by combination of suitable oxidation dye precursors. Currently in vogue besides modified natural shades are, for example, brown shades with pronounced eggplant or copper hues and also brilliant red hues.

Besides creating color effects, oxidation dyes intended for treating human hair must meet increasingly stringent requirements. On the one hand, the dyes must be toxicologically and dermatologically harmless and they must be nonsensitizing. In addition, by combination of suitable developer and coupler components, they must provide a wide range of different color shades.

Furthermore, the hair colorations produced must have good light stability and be resistant to perspiration, permanent wave application, acids, bases and rubbing. At any rate, under currently prevailing conditions such hair colorations must remain stable for at least four to six weeks.

Until now, to provide colorations in the increasingly important red range, 4-aminophenol was in most cases used as the developer. Because of concerns about the physiological compatibility of 4-aminophenol, pyrimidine derivatives have been used as an alternative, but in coloring terms they were not satisfactory. Finally, DE-A 42 34 885 and DE-A 42 34 887 described pyrazole derivatives that give intense red shades.

The aforesaid pyrazoles give highly brilliant and saturated color shades. Because of the high covering ability of the pyrazole dyes, however, as a rule they give very boldly colored hair. However, when it is desired to obtain not the saturated colors but rather the color reflexes of the same color shade that become highly prominent when backlit—persons skilled in the art refer to them as “transparent hair colors”—this requirement cannot be met with the hitherto known pyrazoles. The reason is primarily that transparency effects cannot be achieved simply by using a smaller amount of dye precursors in the dye composition or by diluting the dye compositions more than usual. In most cases, in the case of strongly coloring compounds such methods give very nonuniform and for the customer unsatisfactory results. In practice, this problem manifests itself in varying absorbability of the dyes which could be caused, for example, by a gradually varying hair structure between the undamaged hair base and the damaged hair tips. Nonuniform hair coloring is noted particularly when during hair washing the dyes are washed out more strongly, commensurate with the degree of hair damage. In addition to the usually intact hair base, in the unfavorable case an unnatural, nonuniform and entirely unsatisfactory coloring result is obtained.

The goal of the present invention is to solve the aforesaid problems by providing novel dye precursors for the oxidative coloring system which besides good absorbability and high wash-out resistance at the same time also give rise to “transparent hair colorations”.

We have now found that with certain 4,5-diaminopyrazoles the aforesaid goal can be reached to an outstanding degree.

The invention has for an object novel 4,5-diaminopyrazoles of general formula (I) 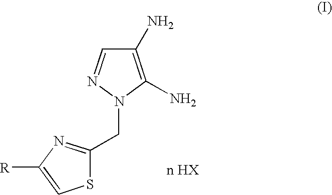
wherein R denotes a straight-chain or branched C₁-C₆-alkyl group, an unsubstituted phenyl group or a singly or multiply substituted phenyl group, wherein the substituents are selected independently of each other from among a halogen atom (F, Cl, Br, I), a C₁-C₆ carboxylate ester group, a straight-chain or branched C₁-C₆-alkoxy group (optionally interrupted by one or two hetero atoms), a hydroxyethoxy group, a dihydroxypropoxy group or a nitrile group.

The compounds of the invention can be prepared, for example, in accordance with the following Scheme 1.

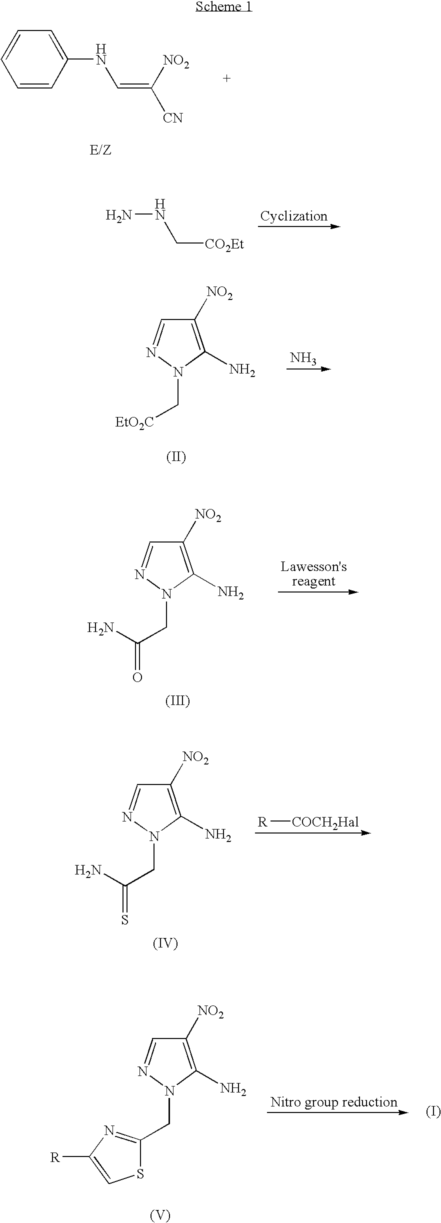

To this end, (2E/Z)-3-anilino-2-nitro-2-propene nitrile, prepared by the method of O.S. Wolfbeis, Chem. Ber. 114 (11), 3471 (1981), is cyclized with ethyl hydrazinoacetate, and the carboxamido group is formed by aminolysis of the ester group. Then, by means of Lawesson's reagent (=2,4-bis(4-methoxyphenyl)-1,3,2,4-dithiadiphosphetan-2,4-dithione), the conversion to the thioamide function is carried out. From this function, the side-chain heterocycles can be formed by reaction with appropriate halogenomethyl aryl ketones. After reduction of the nitro group at the pyrazole ring, the products of the invention can be isolated.

Because of the pronounced oxidation sensitivity of the 4,5-diaminopyrazoles, the compounds are advantageously not isolated as the free bases but as the acid adducts. Most of the resulting salts are mostly oxidation-insensitive permitting easier handling of the compounds.

The acids used can be inorganic or organic, with hydrochloric acid, sulfuric acid, phosphoric acid, boric acid, citric acid and tartaric acid being preferred. Particularly preferred are hydrochloric acid and sulfuric acid.

Examples of suitable compounds of formula (I) are, in particular:

### 1-[(4-phenyl-1,3-thiazol-2-yl)methyl]-4,5-diamino-1H-pyrazole dihydrochloride (1a)

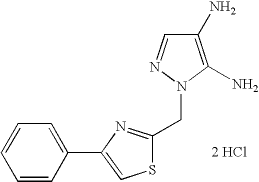

### 1-{[4-(4-aminophenyl-1,3-thiazol-2-yl]methyl]-4,5-diamino-1H-pyrazole trihydrochloride (1b)

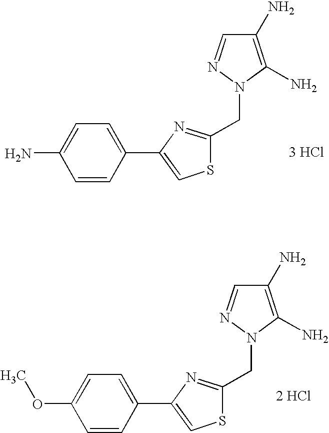

### 1-{[4-(4-Methoxyphenyl)-1,3-thiazol-2-yl)methyl)-4,5-diamino-1H-pyrazole dihydrochloride (1c)

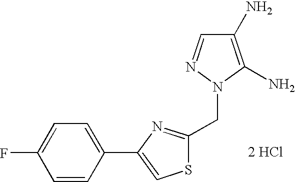

### 1-{[4-(4-Fluorophenyl)-1,3-tiazol-2yl]methyl}-4,5-diamino-1H-pyrazole dihydrochloride (1d)

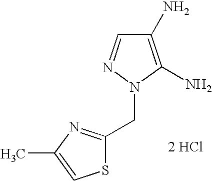

### 1-[(4-Methyl-1,3-thiazol-2-yl)methyl]-4,5-diamino-1H-pyrazole dihydrochloride (1e)

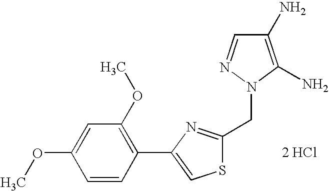

### 1-{[4-(2,4-Dimethoxyphenyl)-1,3-thiazol-2-yl]methyl}-4,5-diamino-1H-pyrazole dihydrochloride (1f)

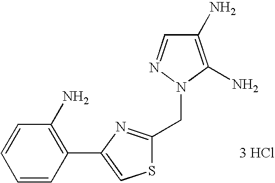

### 1-{[4-(2-Aminophenyl)-1,3-thiazol-2-yl]methyl}-4,5-diamino-1H-pyrazole trihydrochloride (1g)

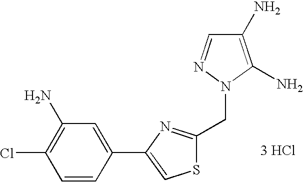

### 1-{[4-(3-Amino-4-chlorophenyl)-1,3-thiazol-2-yl]methyl}-4,5-diamino-1H-pyrazole trihydrochloride (1h)

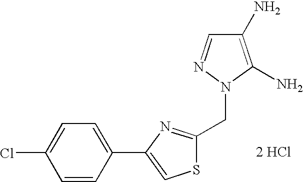

### 1-{[4-(4-Chlorophenyl)-1,3-thiazol-2-yl]methyl}-4,5-diamino-1H-pyrazole dihydrochloride (1i)

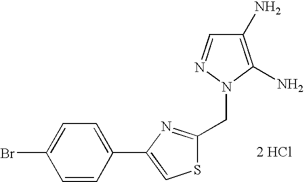

### 1-{[4-(4-Bromophenyl)-1,3-thiazol-2-yl]methyl}-4,5-diamino-1H-pyrazole dihydrochloride (1j)

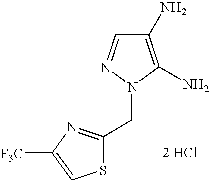

### 1-[(4-trifluoromethyl-1,3-thiazol-2-yl)methyl]-4,5-diamino-1H-pyrazole dihydrochloride (1k)

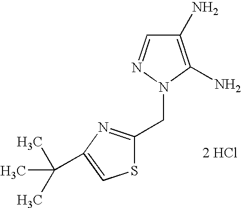

### 1-[(4-t-butyl-1,3-thiazol-2-yl)methyl]-4,5-diamino-1H-pyrazole dihydrochloride (1l)

The compounds of formula (I) are eminently suited as dye precursors in the oxidative system for coloring keratin fibers.

Hence, another object of the present invention is the use of the compounds of formula (I) in colorants for keratin fibers, for example wool, silk or hair, and particularly human hair.

Although the compounds of formula (I) are particularly well suited for dyeing keratin fibers, it is in principle also possible to color with these compounds other natural or synthetic fibers, for example cotton or nylon 66.

The compound of formula (I) can be used alone or in combination with certain known developers and/or couplers commonly employed in oxidative coloring systems for dyeing fibrous materials. In the coloring composition, the 4,5-diaminopyrazoles of formula (I) are contained in an amount from about 0.01 to 20 weight percent and preferably from about 0.1 to 15 weight percent.

Suitable couplers are, in particular: N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 2,4-diamino-1-(3-hydroxypropoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyinole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methyl-phenol, 3-aminophenol, 2-[(3-hydroxyphenyl)amino]acetamide, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro 1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione or the salts thereof.

To produce special nuances or more pleasing shades with stylish reflexes, it may be particularly advantageous to use compounds of formula (I) in combination with additional developers. Suitable developers are para-phenylenediamines, para-aminophenols and other 4,5-diaminopyrazoles or the salts thereof. Particularly suitable developers are the following compounds: 1,4-diaminobenzene (p-phenylenediamine), 1,4-diamino-2-methylbenzene (p-toluylenediamine), 1,4-diamino-2,6-dimethylbenzene, 1,4-diamino-2,5-dimethylbenzene, 1,4-diamino-2,3-dimethylbenzene, 2-chloro-1,4-diaminobenzene, 4-phenylaminoaniline, 4-dimethylaminoaniline, 4-diethylaminoaniline, 4-[di(2-hydroxyethyl)amino]aniline, 4-[(2-methoxyethyl)amino]aniline, 4-[(3-hydroxypropyl)amino]aniline, 1,4-diamino-2-(2-hydroxyethyl)benzene, 1,4-diamino-2-(1-methylethyl)benzene, 1,3-bis[(4-aminophenyl)-(2-hydroxyethyl)amino]-2-propanol, 1,8-bis(2,5-diaminophenoxy)-3,6-dioxaoctane, 4-aminophenol, 4-amino-3-methylphenol, 4-methylaminophenol, 4-amino-2-(aminomethyl)phenol, 4-amino-2-[(2-hydroxyethyl)amino]methylphenol, 4-amino-2-(methoxymethyl)phenol, 4-amino-2-(2-hydroxyethyl)phenol, 5-aminosalicylic acid, 2,5-diaminopyridine, 2,4,5,6-tetraminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-3-methyl-1-phenyl-1H-pyrazole, 4,5-diamino-1-(2-hydroxyethyl)-3-methyl-1H-pyrazole, 1,2-bis(4,5-diamino-1H-pyrazol-1-yl)ethane, 1,4-bis(4,5-diaminopyrazol-1-yl)methyl)benzene, 4,5-diamino-1-(2-methylphenyl)-1H-pyrazole, 4,5-diamino-1-(3-methylphenyl)-1H-pyrazole, 4,5-diamino-1-(4-methylphenyl)-1H-pyrazole, 4,5-diamino-1-(2,4-dimethylphenyl)-1H-pyrazole, 4,5-diamino-1-(2,5-dimethylphenyl)-1H-pyrazole, 4,5-diamino-1-(2-ethylphenyl)-1H-pyrazole, 4,5-diamino-1-(4-isopropylphenyl)-1H-pyrazole, 4,5-diamino-1-(4-methoxyphenyl)-1H-pyrazole, 1-(4-aminophenyl)-4,5-diamino-1H-pyrazole, 1-(4-chlorophenyl)-4,5-diamino-1H-pyrazole, 4,5-diamino-1-(2-pyridinyl)-1H-pyrazole, 2-aminophenol, 2-amino-6-methyl-phenol and 2-amino-5-methylphenol or the salts thereof.

Naturally, the compounds of formula (I) can also be used in combination with common direct anionic, cationic or neutral dyes. The preferred anionic dyes include, for example, disodium 6-hydroxy-5-[(4-sulfophenyl)azo]-2-naphthalenesulfonate (C.I. 15 985; Food Yellow No. 3; FD&C Yellow No. 6), disodium 2,4-dinitro-1-naphthol-7-sulfonate (C.I. 10 316; Acid Yellow No. 1; Food Yellow No. 1), 2-(indan-1,3-dion-2-yl)quinoline-x,x-sulfonic acid (mixture of mono- and disulfonic acid) (C.I. 47 005; D&C Yellow No. 10, Food Yellow No. 13, Acid Yellow No. 3), trisodium 5-hydroxy-1-(4-sulfophenyl)-4-[(4-sulfophenyl)azo]pyrazole-3-carboxylate (C.I. 19 140; Food Yellow No. 4; Acid Yellow No. 23), 9-(2-carboxyphenyl)-6-hydroxy-3H-xanthen-3-one (C.I. 45 350, Acid Yellow No. 73; D&C Yellow No. 8), sodium 5-[(2,4-dinitrophenyl)amino]-2-phenylaminobenzenesulfonate (C.I. 10 385; Acid Orange No. 3), monosodium 4-[(2,4-dihydroxyphenyl)azo]benzenesulfonate (C.I.14 270; Acid Orange No. 6), sodium 4-[(2-hydroxynaphth-1-yl)azo]benzenesulfonate (C.I.15 510; Acid Orange No. 7), sodium 4-[(2,4-dihydroxy-3-[(2,4-dimethylphenyl)azo]phenyl)azo]benzenesulfonate (C.I. 20 170; Acid Orange No. 24), disodium 4-hydroxy-3-[(4-sulfonaphth-1-yl)azo]-1-naphthalenesulfonate (C.I. 14720; Acid Red No. 14), trisodium 6-hydroxy-5-[(4-sulfonaphth-1-yl)azo]-2,4-naphthalenedisulfonate (C.I. 16 255; Ponceau 4R; Acid Red No. 18), trisodium 3-hydroxy-4-[(4-sulfonaphth-1-yl)azo]-2,7-naphthalenedisulfonate (C.I. 16 185; Acid Red No. 27), 8-amino-1-hydroxy-2-(phenylazo)-3,6-naphthalenedisulfonate (C.I. 17 200; Acid Red No. 33), disodium 5-(acetylamino)-4-hydroxy-3-[(2-methylphenyl)azo]-2,7-naphthalenedisulfonate (C.I. 18 065; Acid Red No. 35), disodium 2-(3-hydroxy-2,4,5,7-tetraiododibenzopyran-6-on-9-yl)benzoate (C.I. 45 430; Acid Red No. 51), N-[6-(diethylamino)-9-(2,4-disulfophenyl)-3H-xanthen-3-ylidene]-N-ethylethanaminium hydroxide, inner salt, sodium salt (C.I. 45 100; Acid Red No. 52), disodium 8-[(4-(phenylazo)phenyl)azo]-7-naphthol-1,3-disulfonate (C.I. 27 290; Acid Red No. 73), disodium 2′,4′,5′,7′-tetrabromo-3′-6′-dihydroxyspiro-{isobenzofuran-1(3H), 9′-[9H]xanthen}-3-one (C.I. 45 380; Acid Red No. 87), disodium 2′,4′,5′,7′-tetrabromo-4,5,6,7-tetrachloro-3′,6′-dihydroxyspiro{isobenzofuran-1 (3H), 9′[9H]-xanthen}-3-one (C.I. 45 410; Acid Red No. 92), disodium 3′,6′-dihydroxy-4′,5′-diiodospiroisobenzofuran-1(3H), 9′(9H)-xanthen)-3-one (C.I. 45 425; Acid Red No. 95), disodium (2-sulfophenyl)-di[4-(ethyl((4-sulfophenyl)methyl)amino)phenyl]carbenium, betaine (C.I. 42 090; Acid Blue No. 9; FD&C Blue No. 1), disodium 1,4-bis[(2-sulfo-4-methylphenyl)amino]-9,10-anthraquinone (C.I. 61 570; Acid Green No. 25), bis[4-dimethylamino)phenyl]-3,7-disulfo-2-hydroxynaphth-1-yl)carbenium inner salt, monosodium salt (C.I. 44 090; Food Green No. 4; Acid Green No. 50), bis[4-(diethylamino)phenyl](2,4-disulfophenyl)carbenium inner salt, sodium salt (2:1) (C.I. 42 045; Food Blue No. 3; Acid Blue No. 1), bis[4-(diethylamino)phenyl](5-hydroxy-2,4-disulfophenyl)carbenium inner salt, calcium salt (2:1) (C.I. 42 051; Acid Blue No. 3), sodium 1-amino-4-(cyclohexylamino)-9,10-anthraquinone-2-sulfonate (C.I. 62 045; Acid Blue No. 62), disodium 2-(1,3-dihydro-3-keto-5-sulfo-2H-indol-2-ylidene)-2,3-dihydro-3-keto-1H-indole-5-sulfonate (C.I. 73 015); Acid Blue No. 74), 9-(2-carboxyphenyl)-3-[(2-methylphenyl)amino]-6-[(2-methyl-4-sulfophenyl)amino]xanthylium inner salt, monosodium salt (C.I. 45 190; Acid Violet No. 9), sodium 1-hydroxy-4-[(4-methyl-2-sulfo-phenyl)amino]-9,10-anthraquinone (C.I. 60 730; D&C Violet No. 2; Acid Violet No. 43), bis{3-nitro-4-[(4-phenylamino)-3-sulfophenylamino]-phenyl}sulfone (C.I. 10 410; Acid Brown No. 13), disodium 5-amino-4-hydroxy-6-[(4-nitrophenyl)azo]-3-(phenylazo)-2,7-naphthalenedisulfonate (C.I. 20 470; Acid Black No. 1), 3-hydroxy-4-[(2-hydroxynaphth-1-yl)azo]-7-nitro-1-naphthalenesulfonic acid chromium complex (3:2) (C.I. 15711; Acid Black No. 52), disodium 3-[(2,4-dimethyl-5-sulfophenyl)azo]-4-hydroxy-1-napthalenesulfonate (C.I. 14 700; Food Red No. 1; Ponceau SX; FD&C Red No. 4), tetrasodium 4-(acetylamino)-5-hydroxy-6-{7-sulfo-4-[(4-sulfophenyl)azo]naphth-1-yl)azo}-1,7-naphthalenedisulfonate (C.I. 28 440; Food Black No. 1) and sodium 3-hydroxy-4-(3-methyl-5-keto-1-phenyl-4,5-dihydro-1H-pyrazol-4-ylazo)naphthalenesulfonate chromium complex (Acid Red No. 195).

Preferred cationic dyes include, for example, 9-(dimethylamino)benzo[a]phenoxazin-7-ium chloride (C.I. 51 175; Basic Blue No. 6), di[4-(diethylamino)phenyl][4-(ethylamino)naphthyl]carbenium chloride (C.I. 42 595; Basic Blue No. 7), 3,7-di(dimethylamino)phenothiazin-5-ium chloride (C.I. 52 015; Basic Blue No. 9), di[4-(dimethylamino)phenyl][4-(phenylamino)naphthyl]carbenium chloride (C.I. 44 045; Basic Blue No. 26), 2-{[4-(ethyl-(2-hydroxyethyl)amino]phenyl)azo}-6-methoxy-3-methylbenzothiazolium methylsulfate (C.I. 11 154; Basic Blue No. 41), 8-amino-2-bromo-5-hydroxy-4-imino-6-{[3-(trimethylammonio)phenyl]amino}-1(4H)-naphthalinone chloride (C.I. 56 059; Basic Blue No. 99), bis[4-(dimethylamino)phenyl][4-(methylamino)phenyl]carbenium chloride (C.I. 42 535; Basic Violet No. 1), tris(4-amino-3-methylphenyl)carbenium chloride (C.I. 42 520; Basic Violet No. 2), tris[4-(dimethylamino)phenyl]carbenium chloride (C.I. 42 555; Basic Violet No. 3), 2-[3,6-(diethylamino)dibenzopyranium-9-yl]benzoyl chloride (C.I. 45 170; Basic Violet No. 10), di(4-aminophenyl)(4-amino-3-methylphenyl)carbenium chloride (C.I. 42 510; Basic Violet No. 14), 1,3-bis[(2,4-diamino-5-methylphenyl)azo]-3-methylbenzene (C.I. 21 010; Basic Brown No. 4), 1-[(4-aminophenyl)azo]-7-(trimethylammonio)-2-naphthol chloride (C.I. 12 250; Basic Brown No. 16), 1-[(4-amino-2-nitrophenyl)azo]-7-(trimethylammonio)-2-naphthol chloride (C.I. 12 251; Basic Brown No. 17), 1-[(4-amino-2-nitrophenyl)azo]-7-(trimethylammonio)-2-naphthol chloride (C.I. 12 251; Basic Brown No. 17) [sic], 3,7-diamino-2,8-dimethyl-5-phenylphenazinium chloride (C.I. 50 240; Basic Red No. 2), 1,4-dimethyl-5-{[4-(dimethylamino)phenyl]azo}-1,2,4-triazolium chloride (C.I. 11 055; Basic Red No. 22), 2-hydroxy-1-[(2-methoxyphenyl)azo]-7-(trimethylammonio)naphthalene chloride (C.I. 12 245; Basic Red No 76), 2-{2-[(2,4-dimethoxyphenyl)amino]ethenyl}-1,3,3-trimethyl-3H-indol-1-ium chloride (C.I. 48 055; Basic Yellow No. 11), 3-methyl-1-phenyl-4-{[3-(trimethylammonio)phenyl]azo}pyrazol-5-one chloride (C.I. 12 719; Basic Yellow No. 57) and bis[4-(diethylamino)phenyl]carbenium hydrogen sulfate (1:1) (C.I. 42 0 040; Basic Green No. 1).

To improve color balancing and to produce special shades, the nonionic dyes from the following group have been found to be particularly useful: 1-amino-2-[(2-hydroxyethyl)amino]-5-nitrobenzene (HC Yellow No. 5), 1-(2-hydroxyethoxy)-2-.([(2-hydroxyethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitrophenol, 1-(2-hydroxyethoxy)3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene, 4-[(2,3-dihydroxypropyl)amino]-3-nitro-1-trifluoromethylbenzene (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene (HC Yellow No. 13), 4-[(2-hydroxyethyl)amino]-3-nitrobenzonitrile (HC Yellow No. 14), 4-[(2-hydroxyethyl)amino]-3-nitrobenzamide (HC Yellow No. 15), 1-amino-4-[(2-hydroxyethyl)amino]-2-nitrobenzene (HC Red No. 7), 2-amino-4,6-dinitrophenol, 2-ethylamino-4,6-dinitrophenol, 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-[(2-aminoethyl)amino]-4-(hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro-4-[(2,3-dihy-droxypropyl)amino]-2-nitrobenzene (HC Red No. 10), 5-chloro-1,4-[di(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-methylamino-4-nitrophenol, 2-chloro-6-[(2-hydroxyethyl)amino]-4-nitrophenol, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1,4-bis[(2-hydroxyethyl)amino]-2-nitrobenzene, 1-(2-hydroxyethyl)amino-2-nitro-4-[di(2-hydroxyethyl)amino]benzene (HC Blue No. 2), 1-amino-3-methyl-4-[(2-hydroxyethyl)amino]-6-nitrobenzene (HC Violet No. 1), 4-[ethyl-(2-hydroxyethyl)amino]-1-[(2-hydroxyethyl)amino]-2-nitrobenzene hydrochloride (HC Blue No. 12), 4-[di(2-hydroxyethyl)amino]-1-[(2-methoxyethyl)amino]-2-nitrobenzene (HC Blue No. 11), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene (HC Violet No. 2), 1-methylamino-4-[methyl-(2,3-dihydroxypropyl)amino]-2-nitrobenzene (HC Blue No. 6), 2-[(4-amino-2-nitrophenyl)amino]-5-dimethylaminobenzoic acid (HC Blue No. 13), 1,4-di[2,3-dihydroxypropyl)amino]-9,10-anthraquinone, 1-[(2-hydroxyethyl)amino]-4-methylamino-9,10-anthraquinone (C.I. 61 505, Disperse Blue No. 3), 2-[(2-aminoethyl)amino]-9,10-anthraquinone (HC Orange No. 5), 1-hydroxy-4-[(4-methyl-2-sulfophenyl)amino]-9,10-anthraquinone, 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (C.I. 62 015, Disperse Red No. 11, Solvent Violet No. 26), 1-4-dihydroxy-5,8-bis[(2-hydroxyethyl)amino]-9,10-anthraquinone (C.I. 62 500, Disperse Blue No. 7, Solvent Blue No. 69), 1-[di(2-hydroxyethyl)amino]-3-methyl-4-[(4-nitrophenyl)azo]-benzene (C.I. 11 210, Disperse Red No. 17), 4-[(4-aminophenyl)azo]-1-[di(2-hydroxyethyl)amino]-3-methylbenzene (HC Yellow No. 7), 2,6-diamino-3-[(pyridin-3-yl)azo]-pyridine and 2-{[4-(acetylamino)phenyl]azo}-4-methylphenol (C.I. 11 855; Disperse Yellow No. 3).

Particularly noteworthy among the direct dyes are 2-amino-4,6-dinitrophenol, 2-ethylamino-4,6-dinitrophenol, 2-[(2-hydroxyethyl)amino]-4,6-dinitrophenol and the dyes of general formula (II) 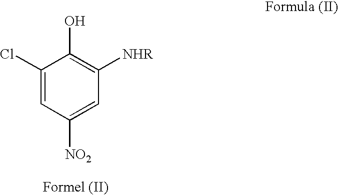
wherein R denotes hydrogen, methyl, ethyl or hydroxyethyl.

The aforedescribed combinations according to the invention of compounds of formula (I) with oxidative hair dye precursors and/or direct dyes are applied for dyeing in an appropriate dye carrier composition.

Another object of the present invention is an agent for oxidative dyeing of hair that is prepared by mixing a dye carrier composition with an oxidant just before use and is characterized in that it contains at least one compound of formula (I) and optionally other dye precursors and/or direct dyes.

The compounds of formula (I) and the dye precursors are contained in this ready-to-use colorant at a total concentration from about 0.01 to 10 weight percent and preferably from 0.2 to 6 weight percent. The total concentration of direct dyes is about 0.1 to 10 weight percent and preferably 0.1 to 5 weight percent.

Besides the colorant, the dye carrier composition can also contain antioxidants, perfume oils, complexing agents, wetting agents, emulsifiers, penetrants, buffering systems, preservatives, thickeners, hair-care agents and other cosmetic additives.

The dye carrier composition for the ready-to-use oxidation hair colorant can be formulated, for example, as a solution, particularly an aqueous or aqueous-alcoholic solution. The particularly preferred formulations, however, are creams, gels or emulsions. The composition consists of a mixture of the dye components and the additives commonly used for such compositions.

Common additives for solutions, creams or gels are, for example, solvents, for example, water, lower aliphatic alcohols, for example ethanol, n-propanol and isopropanol or glycols such as glycerol and 1,2-propylene glycol, moreover wetting agents or emulsifiers from the classes of anionic, cationic, amphoteric or nonionic surface-active substances, such as the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, fatty alkanolamides, ethoxylated fatty esters, furthermore thickeners such as the higher fatty alcohols, starch or cellulose derivatives, moreover vaseline, paraffin oil and fatty acids and also hair-care agents such as cationic resins, lanolin derivatives, cholesterol, pantothenic acid and betaine. The said constituents are used in amounts usually employed for such purposes. Based on the dye carrier composition, the wetting agents and emulsifiers, for example, are used at a concentration from about 0.5 to 30 weight percent, the thickeners in an amount from about 0.1 to 25 weight percent and the hair-care agents at a concentration from about 0.1 to 5.0 weight percent.

The ready-to-use hair colorant of the invention is prepared by mixing the dye carrier composition with a liquid oxidant just before use.

Suitable oxidants are primarily hydrogen peroxide or the addition compounds thereof to urea, melamine or sodium bromate in the form of a 1 to 12% and preferably a 6% aqueous solution. Hydrogen peroxide is particularly preferred.

The dye carrier composition and the oxidant are mixed with one another in a weight ratio of 5:1 to 1:3, a weight ratio of 1:1 to 1:2 being particularly preferred

Upon mixing the preferably alkaline dye carrier composition with the mostly acidic oxidant, the pH of the ready-to-use hair colorant of the invention assumes a value that depends on the amount of alkali in the dye carrier composition, on the amount of acid in the oxidant and on the mixing ratio. The ready-to-use hair colorant has a pH from about 3 to 11 and preferably from 6 to 10.5.

To adjust the pH of the dye carrier composition and of the oxidant, a dilute organic or inorganic acid can be used, for example phosphoric acid, ascorbic acid or lactic acid, or an alkali, for example monoethanolamine, triethanolamine, 2-amino-2-methyl-1-propanol, ammonia, sodium hydroxide, potassium hydroxide or tris(hydroxymethyl)aminomethane, depending on the pH desired.

After the afore-described dye carrier composition has been mixed with the oxidant, an amount of the resulting ready-to-use mixture sufficient for the hair-dyeing treatment is applied to the hair, in general about 60 to 200 grams, depending on the hair fullness.

The hair colorant of the invention is allowed to act on the hair for about 10 to 45 minutes at 15 to 50° C. and preferably for 30 minutes at 40° C. after which the hair is rinsed with water. After this rinsing, the hair is optionally washed with a shampoo and optionally post-rinsed with a dilute weak organic acid, for example citric acid or tartaric acid. The hair is then dried.

The following examples will explain the subject matter of the invention in greater detail without limiting its scope to said examples.

### EXAMPLES

### Example 1

### Preparation of Thiazolylmethylpyrazoles of Formula (I)

### Step 1: Ethyl (5-amino-4-nitro-1H-pyrazol-1-yl)acetate (II)

To a suspension of 37.75 g (200 mmol) of (2-E/Z)-3-anilino-2-nitro-2-propene nitrile and 46.36 g (300 mmol) of ethyl hydrazinoacetate hydrochloride in 250 mL of anhydrous tetrahydrofuran was added 155 mL of diisopropylethylamine at 4° C. with agitation. The mixture was allowed to agitate at this temperature for one hour, after which 130 mL of ethanol was added and the mixture was heated at reflux for 18 hours. After cooling, about 200 mL was distilled off, the residue was poured into 500 mL of saturated sodium hydrogen carbonate solution and the resulting mixture was extracted twice with 500-mL portions of ethyl acetate. The combined ethyl acetate phases were dried over anhydrous magnesium sulfate and concentrated to about 100 mL after which hexane was added to incipient crystallization. The product was allowed to crystallize and was then suction-filtered off and dried at 40° C. under vacuum.

Yield: 19.8 g (46%)

Mᵣ: 214.18 (C₇H₁₀N₄O₄)

MS (APCI⁺): 215 [M+H]⁺

¹H-NMR (DMSO-d₆): 7.93 (s, pyrazole-H); 7.59 (s, NH₂); 4.89 (s, NCH₂); 4.16 (q, J=7.1, OCH₂); 1.22 (t, J=7.1, CH₃).

### Step 2: (5-Amino-4-nitro-1H-pyrazol-1-yl)acetamide (III)

To a solution of 17.3 g (80 mmol) of ethyl (5-amino-4-nitro-1H-pyrazol-1-yl)acetate from Step 1 in 100 mL of methanol was added 100 mL of 4N methanolic ammonia solution. After an agitation period of about 5 minutes, crystallization set in. The mixture was allowed to agitate overnight after which it was suction-filtered, and the filter cake was washed with a small amount of cold methanol and dried at 40° C. under vacuum.

Yield: 13.6 (99%) colorless crystals

Mᵣ: 185.14 (C₅H₇N₅O₃)

MS (APCI⁺): 186 [M+H]⁺

¹H-NMR (DMSO-d₆): 7.88 (s, pyrazole-H); 7.50, 7.47, 7.28 (3s, 2 NH₂); 4.60 (s, CH₂).

### Step 3: (5-Amino-4-nitro-1H-pyrazol-1-yl)thioacetamide (IV)

To a solution of 10 g (54 mmol) of the (5-amino-4-nitro-1H-pyrazol-1-yl)acetamide from Step 2 in 150 mL of tetrahydrofuran was added 26.2 g (65 mmol) of Lawesson's reagent at room temperature, and the reaction mixture was heated at reflux. After 1 hour, the reaction mixture was cooled, filtered through silica gel and evaporated to dryness. The residue was taken up with 100 mL of methylene chloride and the mixture was allowed to agitate at room temperature for 30 min which caused the product to crystallize. Suction-filtration of the residue and drying under vacuum at 40° C. gave 9.9 g of a pale-yellow powder.

Mᵣ: 201.20 (C₅H₇N₅O₂S)

MS (APCI⁺): 202 [M+H]⁺

¹H-NMR (CD₃OD): 7.91 (s, pyrazole-H)); 4.97 (s, CH₂).

### Step 4: 4-Nitro-5-amino-1-thiazolyl compounds of formula (V)

To a solution of 1 g (5 mmol) of the (5-amino-4-nitro-1H-pyrazol-1-yl)thioacetamide from Step 3 in 20 mL of tetrahydrofuran/isopropanol 1:1 was added 6 mmol of halogeno-methylaryl alkyl ketone, and the mixture was heated at reflux for 5 hours. The reaction mixture was cooled and evaporated and then extracted using a two-phase system consisting of 20 mL of saturated sodium hydrogen carbonate solution and 20 mL of ethyl acetate. The organic phase was separated and dried with a small amount of magnesium sulfate. It was then concentrated to about 5 mL, and the product was made to crystallize by addition of ether.

Thin-layer chromatography showed the compounds to be uniform. After drying under vacuum at 40° C., the nitro compounds of formula (V) were obtained in pure form. In the following are given the analytical data for nitro compounds of formula (V) chosen as examples.

a) R=phenyl [C₁₃H₁₁N₅O₂S; Mᵣ=301.32]

MS (APCI⁺): 302 [M+H]⁺

¹H-NMR (CDCl₃): 7.89 (s, 1H); 7.86-7.82 (m, 2H); 7.53-7.39 (m, 4H); 6.76 (br. s, NH₂); 5.48 (s, CH₂).

b) R=4-O-methylphenyl [C₁₄H₁₃N₅O₃S; Mᵣ=331.35]

MS(ESI⁺): 332 [M+H]⁺

¹H-NMR (DMSO-d₆): 8.0 (s, 1H); 7.89 (s, 1H), 7.84, 6.98 (AB, J=8.8, 4H); 7.71 (s, 2H); 5.61 (s, CH₂); 3.78 (s, OMe)

c) R=methyl

¹H-NMR (DMSO-d₆): 7.98 (s, pyrazole-H); 7.69 (s, NH₂), 7.24 (q, J=1.0, thiazole-H); 5.52 (s, CH₂); 2.34 (d, J=1.0, Me)

d) R=2-nitrophenyl [C₁₃H₁₀N₆O₄S; Mᵣ=346.32]

MS(ESI⁺): 347 [M+H]⁺

¹H-NMR (DMSO-d₆): 8.0, 7.98 (2s, 2H); 7.89-7.86, 7.79-7.56 (2 m, 6H); 5.57 (s, CH₂).

### Step 5: Synthesis of the thiazolylmethylpyrazoles of formula (I)

A solution of 1 mmol of the nitro compound of formula (V) from Step 4 was hydrogenated in 5 mL of a 1:1 mixture of tetrahydrofuran/isopropanol for 6 hours on 50 mg of 10% Pd/C at 9 bar hydrogen pressure. After the reduction, the mixture was filtered through silica gel (Hyflo SuperCel) directly into 2 mL of 3.3 M ethanolic hydrochloric acid, and the mixture was allowed to agitate in an ice bath. The products that precipitated were colorless to pale-pink. The mixture was suction-filtered, and the products were dried under vacuum with exclusion of oxygen. The yield amounted to 60-85%.

In the following are given the analytical data for the thiazolylmethylpyrazoles of formula (I) chosen as examples.

1a) R=phenyl [hydrochloride: C₁₃H₁₃N₅S.xHCl; Mᵣ=271.34.xHCl]

MS (APCI⁺): 272 [M+H]⁺

1b) R=4-aminophenyl [hydrochloride: C₁₃H₁₄N₆S.xHCl;

Mᵣ=286.35.xHCl]

MS (APCI⁺): 287 [M+H]⁺, 205

¹H-NMR (D₂O): 7.93, 7.46 (AB, J=8.7, 4H); 7.82 (s, 1H); 7.55 (s, 1H); 5.56 ppm (s, CH₂)

1c) R=4-methoxyphenyl [hydrochloride: C₁₄H₁₅N₅OS.xHCl; Mᵣ=301.37.xHCl]

MS(ESI⁺): 302 [M+H]⁺

¹H-NMR (D₂O): 7.64, 6.95 (AB, J=8.9, 4H); 7.55 (s, 1H); 7.53 (s, 1H); 5.52 (s, CH₂); 3.77 ppm (s, OMe)

1d) R=4-fluorophenyl [hydrochloride: C₁₃H₁₂FN₅S.xHCl; Mᵣ=289.33.xHCl]

MS (APCI⁺): 290 [M+H]⁺, 259

1e) R=methyl [hydrochloride:C₈H₁₁N₅S.xHCl; Mᵣ=209.27.xHCl]

MS(ESI⁺): 210 [M+H]⁺

¹H-NMR (D₂O): 7.55 (s, 1H); 7.37 (q, J=1.0, 1H); 5.62 (s, CH₂); 2.41 ppm (d, J=1.0, Me)

1f) R=2,4-dimethoxyphenyl [hydrochloride: C₁₅H₁₇N₅O₂S.xHCl; Mᵣ=331.39.xHCl]

MS (APCI⁺): 332 [M+H]⁺, 301, 276, 250

1g) R=2-aminophenyl [hydrochloride: C₁₃H₁₄N₆S.xHCl; Mᵣ=286.35.xHCl]

MS(ESI⁺): 287 [M+H]⁺

¹H-NMR (D₂O): 7.95 (s, pyrazole-H); 7.87-7.84 (m, thiazole-H); 7.56-7.42 (m, 4H); 5.60 ppm (s, CH₂)

1h) R=3-amino-4-chlorophenyl [hydrochloride: C₁₃H₁₃ClN₆S.xHCl; Mᵣ=323.82.xHCl]

MS (APCI⁺): 321, 323 [M+H]⁺.

### Example 2

### Hair Colorants with a Basic pH

0.25 mmol of the pyrazoles of formula (I) indicated in the following Tables 1-8 was dissolved in 10 mL of the basic composition given hereinbelow.

| | | |
|---|---|---|
| 80.00 | g | of ethanol |
| 100.00 | g | of sodium lauryl ether sulfate, 28% aqueous solution |
| 90.00 | g | of ammonia, 25% aqueous solution |
| 3.00 | g | of ascorbic acid |
| 4.00 | g | of sodium sulfite |
| to 1000.00 | g | demineralized water |

In the same manner, 0.25 mmol of the couplers indicated in the following Tables 1-8 was dissolved in 10 mL of the basic composition.

Just before use, 10 g of pyrazole solution was mixed with 10 g of coupler solution. To this mixture was then added 20 g of a 6% hydrogen peroxide solution followed by stirring which gave the ready-to-use coloring solution.

The ready-to-use oxidation hair colorants thus obtained were then applied to bleached animal hair. After an exposure time of 30 min at 40° C., the hair strands were washed with a shampoo gentle to the dye, rinsed with water and dried. The resulting color shades had readily noticeable transparency. The corresponding L*a*b* values are collected in the following Tables 1-8.

TABLE 1
Developer = 1-[(4-phenyl-1,3-thiazol-2-yl)methyl]-4,5-diamino-1H-
pyrazole dihydrochloride (1a)

| Coupler | L | a | b | Color |
|---|---|---|---|---|
| 3-Aminophenol | 38.44 | 36.67 | 19.99 | red-violet |
| 5-Amino-2-methylphenol | 50.30 | 39.89 | 42.33 | orange |
| 2-(2,4-Diaminophenoxy)- | 26.82 | 31.23 | 2.06 | violet |
| ethanol × 2 HCl | | | | |
| 3-Dimethylaminophenylurea | 32.56 | 20.94 | −25.81 | blue |

TABLE 2
Developer = 1-{[4-(4-aminophenyl)-1,3-thiazol-2-yl]methyl}-4,
5-diamino-1H-pyrazole trihydrochloride (1b)

| Coupler | L | a | b | Color |
|---|---|---|---|---|
| 3-Aminophenol | 33.96 | 35.54 | 17.61 | red-violet |
| 5-Amino-2-methylphenol | 48.74 | 42.31 | 42.95 | orange |
| 2-(2,4-Diaminophenoxy)- | 26.41 | 30.54 | 1.54 | violet |
| ethanol × 2 HCl | | | | |
| 3-Dimethylaminophenylurea | 29.03 | 20.70 | −26.44 | blue |

TABLE 3
Developer = 1-{[4-(4-methoxyphenyl)-1,3-thiazol-2-yl]methyl}-4,
5-diamino-1H-pyrazole dihydrochloride (1c)

| Coupler | L | a | b | Color |
|---|---|---|---|---|
| 3-Aminophenol | 44.45 | 33.90 | 18.43 | red-violet |
| 5-Amino-2-methylphenol | 56.03 | 36.89 | 42.12 | orange |
| 2-(2,4-Diaminophenoxy)- | 32.51 | 30.50 | 2.53 | violet |
| ethanol × 2 HCl | | | | |
| 3-Dimethylaminophenylurea | 39.39 | 19.81 | −26.36 | blue |

TABLE 4
Developer = 1-{[4-(4-fluorophenyl)-1,3-thiazol-2-yl]methyl}-4,
5-diamino-1H-pyrazole dihydrochloride (1d)

| Coupler | L | a | b | Color |
|---|---|---|---|---|
| 3-Aminophenol | 42.07 | 35.04 | 20.02 | red-violet |
| 5-Amino-2-methylphenol | 55.24 | 40.35 | 45.24 | orange |
| 2-(2,4-Diaminophenoxy)- | 29.45 | 31.80 | 3.17 | violet |
| ethanol × 2 HCl | | | | |
| 3-Dimethylaminophenylurea | 35.89 | 21.56 | −27.26 | blue |

TABLE 5
Developer = 1-[(4-methyl-1,3-thiazol-2-yl)methyl]-4,5-diamino-1H-
pyrazole dihydrochloride (1e)

| Coupler | L | a | b | Color |
|---|---|---|---|---|
| 3-Aminophenol | 34.13 | 40.31 | 20.95 | red-violet |
| 5-Amino-2-methylphenol | 48.92 | 44.70 | 46.64 | orange |
| 2-(2,4-Diaminophenoxy)- | 25.19 | 34.96 | 4.32 | violet |
| ethanol × 2 HCl | | | | |
| 3-Dimethylaminophenylurea | 26.89 | 26.28 | −27.77 | blue |

TABLE 6
Developer = 1-{[4-(2,4-dimethoxyphenyl)-1,3-thiazol-2-yl]methyl}-
4,5-diamino-1H-pyrazole dihydrochloride (1f)

| Coupler | L | a | b | Color |
|---|---|---|---|---|
| 3-Aminophenol | 50.44 | 36.12 | 18.51 | red-violet |
| 5-Amino-2-methylphenol | 60.49 | 33.89 | 33.62 | orange |
| 2-(2,4-Diaminophenoxy)- | 38.18 | 29.19 | 7.31 | violet |
| ethanol × 2 HCl | | | | |
| 3-Dimethylaminophenylurea | 41.91 | 19.34 | −25.56 | blue |

TABLE 7
Developer = 1-{[4-(2-aminophenyl)-1,3-thiazol-2-yl]methyl}-
4,5-diamino-1H-pyrazole trihydrochloride (1g)

| Coupler | L | a | b | Color |
|---|---|---|---|---|
| 3-Aminophenol | 32.44 | 36.75 | 18.81 | red-violet |
| 5-Amino-2-methylphenol | 45.02 | 43.73 | 41.33 | orange |
| 2-(2,4-Diaminophenoxy)- | 23.50 | 30.27 | 2.67 | violet |
| ethanol × 2 HCl | | | | |
| 3-Dimethylaminophenylurea | 25.25 | 20.78 | −24.20 | blue |

TABLE 8
Developer = 1-{[4-(3-amino-4-chlorophenyl)-1,3-thiazol-2-yl]methyl}-
4,5-diamino-1H-pyrazole trihydrochloride (1h)

| Coupler | L | a | b | Color |
|---|---|---|---|---|
| 3-Aminophenol | 48.93 | 36.51 | 20.57 | red-violet |
| 5-Amino-2-methylphenol | 58.84 | 34.52 | 40.39 | orange |
| 2-(2,4-Diaminophenoxy)- | 38.25 | 26.95 | 3.27 | violet |
| ethanol × 2 HCl | | | | |
| 3-Dimethylaminophenylurea | 45.30 | 17.35 | −21.56 | blue |

Unless otherwise indicated, all percentages given in the present patent application are by weight.

1. 4,5-Diaminopyrazoles of general formula (I) 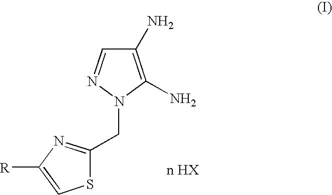
wherein R denotes a straight-chain or branched C₁-C₆-alkyl group, an unsubstituted phenyl group or a singly or multiply substituted phenyl group wherein the substituents are selected independently of each other from among a halogen atom, a C₁-C₆ carboxylate ester group, a straight-chain or branched C₁-C₆-alkoxy group, a straight-chain or branched C₁-C₆-alkoxy group interrupted by one or two hetero atoms, a hydroxyethoxy group, a dihydroxypropoxy group or a nitrile group.

2. 4,5-Diaminopyrazole according to claim 1, characterized in that it is selected from among 1-[(4-phenyl-1,3-thiazol-2-yl)methyl]-4,5-diamino-1H-pyrazole dihydrochloride, 1-{[4-(4-aminophenyl)-1,3-thiazol-2-yl]methyl}-4,5-diamino-1H-pyrazole trihydrochloride, 1-{[4-(4-methoxyphenyl)-1,3-thiazol-2-yl]methyl}-4,5-diamino-1H-pyrazole dihydrochloride, 1-{[4-(4-fluorophenyl)-1,3-thiazol-2-yl]methyl}-4,5-diamino-1H-pyrazole dihydrochloride, 1-[(4-methyl-1,3-thiazol-2-yl)methyl]-4,5-diamino-1H-pyrazole dihydrochloride, 1-{[4-(2,4-dimethoxyphenyl)-1,3-thiazol-2-yl]methyl}-4,5-diamino-1H-pyrazole dihydrochloride, 1-{[4-(2-aminophenyl)-1,3-thiazol-2-yl]methyl}-4,5-diamino-1H-pyrazole trihydrochloride, 1-{[4-(3-amino-4-chlorophenyl)-1,3-thiazol-2-yl]methyl}-4,5-diamino-1H-pyrazole trihydrochlo-ride, 1-{[4-(4-chlorophenyl)-1,3-thiazol-2-yl]methyl}4,5-diamino-1H-pyrazole dihydrochloride, 1-{[4-(4-bromophenyl)-1,3-thiazol-2-yl]methyl}4,5-diamino-1H-pyrazole dihydrochlo-ride, 1-[(4-trifluoromethyl-1,3-thiazol-2-yl)methyl]-4,5-diamino-1H-pyrazole dihydrochloride and 1-[(4-t-butyl-1,3-thiazol-2-yl)methyl]-4,5-diamino-1H-pyrazole dihydrochloride.

3. Dye carrier composition based on oxidation dye precursors, characterized in that it contains at least one 4,5-diaminopyrazole of general formula (I) according to claim 1.

4. Dye carrier composition according to claim 3, characterized in that it contains the 4,5-diaminopyrazole of general formula (I) in an amount from 0.01 to 20 weight percent.

5. Dye carrier composition according to claim 3, characterized in that it contains additionally at least one coupler and/or one additional developer and/or a direct dye.

6. Agent for coloring keratin fibers obtained by mixing a dye carrier composition with an oxidant, characterized in that a dye carrier composition according to claim 3 is used.

7. Agent according to claim 6, characterized in that the oxidant is hydrogen peroxide.

8. Agent according to claim 6, characterized in that the dye carrier composition and the oxidant are mixed in a weight ratio of 5:1 to 1:3.

9. Agent according to claim 6, characterized in that it is a hair colorant.

10. Method for producing 4,5-diaminopyrazoles of formula (I) according to claim 1, characterized in that (2E/Z)-3-anilino-2-nitro-2-propene nitrile is cyclized with ethyl hydra-zinoacetate, after which the carboxamido group is formed by aminolysis and then, by use of Lawesson's reagent, the conversion to the thioamide function is carried out, the side-chain heterocycles being formed from said function by reaction with the appropriate halogenomethyl aryl ketone, and finally the nitro group on the pyrazole ring is reduced.
